(19) United States
(12) Patent Application Publication
(10) Pub. No.: US 2003/0073247 A1
(43) Pub. Date: Apr. 17, 2003

(54) Method for monitoring dynamic particle pollution in an etching chamber

(76) Inventors: Ming-Yu Lin, Hsinchu (TW); Wei Ming Chen, Hsinchu (TW); Yen-Chih Huang, Hsinchu (TW); Shih-Feng Lin, Hsinchu (TW)

Correspondence Address: J.C. Patents, Inc., Suite 250, 4 Venture, Irvine, CA, 92618, US

(21) Appl. No.: 10/017,806

(22) Filed: Oct. 30, 2001

(30) Foreign Application Priority Data
Oct. 15, 2001 (TW) 90125364

(51) Int. Cl. G01N 33/00

(52) U.S. Cl. 436/183; 216/41; 216/2; 430/313

(57) ABSTRACT

A method for monitoring dynamic particle pollution in an etching chamber is provided. The method is described as follows. A bare wafer is positioned in an etching machine. Subsequently the bare wafer is transferred to a main etching chamber. Then, the plasma power source is turned on to perform an etching process on the photoresist. After that, the number of particles fallen on the bare wafer is counted to determine the polluted situation for the etching machine.

### CROSS-REFERENCE TO RELATED APPLICATION

This application claims the priority benefit of Taiwan application serial no. 90125364, filed Oct. 15, 2001.

### BACKGROUND OF THE INVENTION

1. Field of Invention

The present invention relates to a method for monitoring the conditions of an etching machine. More particularly, the present invention relates to a method of monitoring dynamic particle pollution in an etching chamber.

2. Description of Related Art

Etching process is one of the most important steps of semiconductor manufacturing process. Generally, the device pattern on the mask is transferred onto the photoresist by photolithography. Subsequently, by using the etching process the device pattern is then transferred onto the film. The film after photolithography and etching becomes one of semiconductor devices. Taking a MOS or a CMOS process as the example, the film includes SiO₂, Si₃N₄, polysilicon, Al alloy, phosphosilicate (PSG) or the like. That is to say, almost all the materials forming the semiconductor devices must go through the processes of film deposition, photolithography, and etching to complete the manufacture of the semiconductor devices.

The etching technique widely used in the semiconductor manufacturing process has two kinds: one is a wet etching, another is a dry etching, wherein the dry etching is performed by the physical phenomena of the particle bombardment. For example, a plasma dry etching process uses the plasma to decompose the reactive gas molecules into reactive ions, and the reactive ions further chemically react with the films to produce the volatile materials. Subsequently, the volatile materials are evacuated from the etching chamber.

After the etching process has been performed for a while, the particles adhere to the interior walls of the etching chamber. Particle contamination will adversely affect the critical dimensions of devices and reduce the yield. Therefore, it is very important to monitor the particle contamination level in the etching chamber.

In the conventional monitoring methods, a blank wafer, a “bare wafer”, or a product are used for monitoring particle pollution in an etching chamber. However, the conventional monitoring methods are confronted with issues.

When a blank wafer or a bare wafer is used for monitoring particle pollution in an etching chamber, only the actual transportation of products during the etching process is, the actual etching process is not actually performed. If the etching process is actually performed, the blank wafer or the bare wafer can not be reused. This causes the increase of the cost. On the other hand, because the etching process is not actually performed, the products contaminated by the particle pollution in the etching chamber during the manufacturing process can not be precisely to know the actual contamination by particles in the actual fabrication processes.

In the case of directly monitoring products for understanding particle contamination level in the etching chamber, the production line has to be stop while the defect products are founded. It usually has a time difference of at least a half day from the products in the etching chamber at the beginning to the defect products being found. It also take about another half day for monitoring the particles fallen on the products after the production line is stop. As a result, the contamination level of the particle inside the etching chamber during the etching process can not show by using the conventional monitoring method. In addition, because the production line has to stop in the conventional monitoring method, the yield and the throughput are affected.

### SUMMARY OF THE INVENTION

It is therefore an object of the present invention to provide a method for monitoring dynamic particle pollution in an etching chamber. In the present invention, the particles fallen on the wafer can be monitored in time. When the critical particle contamination level is once reached, the etching machine can be cleaned immediately.

It is another object of the present invention to provide a method for monitoring dynamic particle pollution in an etching chamber. In the present invention, the particle pollution inside an etching chamber can be efficiently monitored.

It is a further object of the present invention to provide a method for monitoring dynamic particle pollution in an etching chamber. In the present invention, the wafers used for monitoring particle pollution can be recycled. As a result, the cost is reduced.

The present invention provides a method for monitoring dynamic particle pollution in an etching chamber comprising the following steps. Firstly, a bare wafer is disposed in an etching machine, and the bare wafer is then transferred to a main etching chamber. Subsequently, the plasma power source is turned on to perform an etching for the photoresist formed on the bare wafer. After that, the number of the particles fallen on the bare wafer are monitored to detect the particle contamination level inside the etching machine.

According to the present invention, before the bare wafer is transferred to a main etching chamber, the bare wafer is transferred to a vacuum chamber and a pre-alignment chamber in sequence. The bare wafer is not only transferred but also etched by performing an etching process during the etching process. Therefore, the monitoring method used in the present invention can more effectively reflect the particle contamination level inside the etching chamber during the actual manufacturing process than the conventional monitoring method.

Because the bare wafer used for monitoring particle pollution in the etching chamber can be recycled, the cost is greatly reduced in the present invention.

In addition, it only takes 10 minutes for performing an etching process and monitoring the number of particle pollution fallen on the bare wafer in the present invention. Before the manufacturing semiconductor device process, the number of the particles fallen on the bare wafer is monitored. On the other hand, the particle pollution in the etching chamber can be immediately monitored upon the defect products are found. If it is found that there are some problems in the etching chamber, the etching machine is immediately cleaned.

The method for monitoring particle pollution by using the bare wafer can efficiently show the polluted situation for the etching machine, thereby further increasing the throughput and yield.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a cross-sectional view of the wafer with the particles fallen thereon before the etching process.

FIG. 2 is a cross-sectional view of the wafer with the particles fallen thereon after the etching process.

FIG. 3 is a flow chart, illustrating the successive steps in monitoring dynamic particle pollution in an etching chamber according to a preferred embodiment of the present invention.

### DESCRIPTION OF THE PREFERRED EMBODIMENTS

The present invention method uses the bare wafer for simulating the transporting procedure in the etching process and actually performing the etching process. The monitored result of the present invention can effectively reflect the actual particle contamination level inside the etching chamber during the manufacturing process.

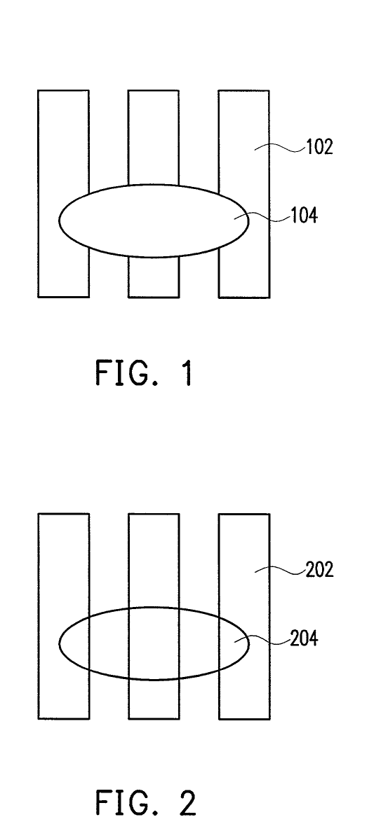

Before the procedure for monitoring dynamic particle pollution in an etching chamber disclosed in the present invention, the product is positioned in a defect monitor device for monitoring the number of particles. If the number of particles fallen on the products exceeds by a certain amount, the quality of the product is not good. The products covered with contaminant particles thereon are then examined by a scanning electron microscope (SEM). After the scanning step, there are two situations can be found as shown in FIGS. 1 and 2.

Referring to FIG. 1, the portion of the product pattern 102 covered by particles 104 does not appear in the final product pattern 102. That is, the particles 104 fall onto the wafers before the etching process, and the fallen particles 104 would act as the etching mask during the etching process. Therefore, the product pattern 102 covered by particles 104 does not appear in the final product pattern 102 after the etching process as shown in FIG. 1.

Referring to FIG. 2, the portion of the product pattern 202 covered by particles 204 apparently appears in the final product pattern 202. That is, the particles 204 fall onto the wafers after the etching process, and the profile of the fallen particles 204 will be conformal with the profile of the final product pattern 202. Therefore, the portion of the product pattern 202 covered by particles 204 apparently appear in the final product pattern 202 as shown in FIG. 2.

Because the particle polluted product which is found by a scanning electron microscope usually has the shape as shown in FIG. 1, it may be concluded that the particles fall onto the wafers while the plasma power source is turned on. That is to say, the particles have fallen onto the wafers before the etching step is started.

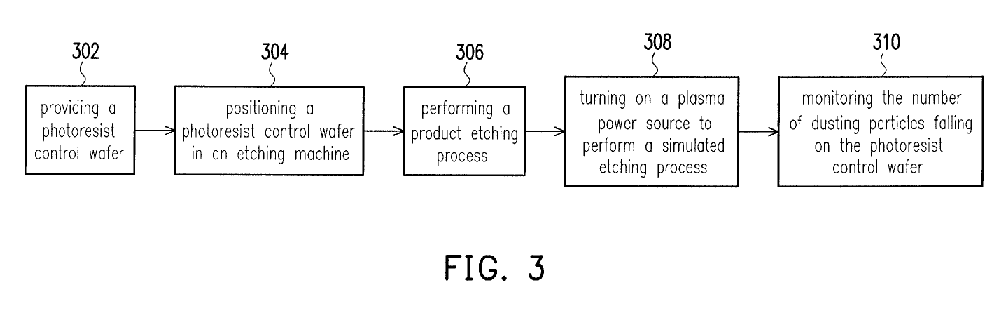

The present invention provides a method for monitoring dynamic particle pollution in an etching chamber. FIG. 3 is a flow chart, illustrating the successive steps in monitoring dynamic particle pollution in an etching chamber according to a preferred embodiment of the present invention. The successive steps in monitoring dynamic particle pollution in an etching chamber will be described as follows.

Step 302: A photoresist is coated on a wafer to form the bare wafer. The photoresist is, for example, a positive photoresist or a negative photoresist, and is formed by, for example, spin coating.

Step 304: The bare wafer is positioned in an etching machine to perform an etching process. The etching machine is, for example, a silicon nitride etching machine, a silicon oxide etching machine, a silicon oxynitride etching machine, a polysilicon etching machine or a metal etching machine.

Step 306: The product etching process is performed. The bare wafer is transported from the port of the etching machine to the main etching chamber. In the transporting procedure, the bare wafer is transported from the port to the vacuum chamber, then from the vacuum chamber to the pre-alignment chamber, then from the pre-alignment chamber to the main etching chamber.

Step 308: The plasma power source is turned on to perform an etching process for the photoresist formed on the bare wafer.

Step 310: The etched the bare wafer is positioned in the defect monitor device to monitor the number of particles thereon. The actual situation for the etching chamber can read on the number of particles fallen on the bare wafer.

The number of particles fallen on the bare wafer is monitored by performing the step 302 through the step 310. The number of particles can simulate and reflect the actual particle contamination level in the etching chamber during the actual etching process for product.

The product covered with the particles thereon usually has the image as shown in FIG. 1. It is proved that the particles fall onto the wafers while the plasma power source is turned on. That is to say, the particles have fallen onto and polluted the wafers before the etching step starts. Therefore, after the plasma power source is turned on, the etching time of the etching process in the step 308 can be shorter than the actual etching time. That is to say, the actual etching time is about 90 seconds, but the etching time of the etching process is set to about ⅙ to 1/10 of the actual etching time, for example, 9 seconds to 15 seconds, and preferably 10 seconds. After that, the etched bare wafer is positioned in a defect monitor device to count the number of particles fallen on the bare wafer. The number of particle monitored by the defect monitor device can determine the particle contamination level in the actual etching process. On the other hand, because it takes only about 10 minutes for performing an etching process and counting the number of particle pollution fallen on the bare wafer, the conditions of the etching machine can be effectively and quickly detected.

As the number of particles fallen on the bare wafer increases, the yield decreases. If the number of particle exceeds by a certain amount, the products have defects. Therefore, before the actual manufacturing process, the step 302 to the step 310 is sequentially performed for monitoring particle pollution in an etching chamber. If the number of particles exceeds by a certain amount, the etching machine should be cleaned immediately. However, if the number of particle does not exceed by a certain amount, the actual manufacturing process can start. The method of the present invention for monitoring particle pollution in an etching chamber is used for avoiding the interference between the wafers and contaminant particle pollution, thereby increasing the yield. On the other hand, if the products are found to have defects, the procedure of the present invention for monitoring particle pollution in an etching chamber is conducted for effectively and quickly detecting the conditions of the etching machine. If the number of particles exceeds by a certain amount, the etching machine should be cleaned immediately.

In addition, the present invention uses a bare wafer instead of a blank wafer, a bare wafer or a product for monitoring particle pollution in an etching chamber. The method of monitoring particle pollution in an etching chamber according to the present invention not only simulates the transporting procedure in an etching process but also actually executes the etching process.

In the present invention, the bare wafer used for monitoring particle pollution in an etching chamber can be recycled simply by stripping off the photoresist formed thereon. As a result, the cost is greatly reduced.

The method for monitoring particle pollution by using the bare wafer can effectively and quickly reflect the actual conditions of the etching machine, thereby further increasing the throughput and the yield.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method of monitoring dynamic particle pollution in an etching chamber, comprising the steps of:
providing a bare wafer coated with a photoresist;
positioning the bare wafer in an etching machine and performing an etching process on the photoresist; and
counting the amount of the particles on the etched wafer so as to determine polluted situation for the etching machine.

2. The method of claim 1, wherein the etching is carrier out for 9 seconds to 15 seconds.

3. The method of claim 1, wherein the etching machine is a silicon nitride etching machine.

4. The method of claim 1, wherein the etching machine is a silicon oxide etching machine.

5. The method of claim 1, wherein the etching machine is a silicon oxynitride etching machine.

6. The method of claim 1, wherein the etching machine is a polysilicon etching machine.

7. The method of claim 1, wherein the etching machine is a metal etching machine.

8. The method of claim 1, wherein the step of performing the etching process comprises:
transporting the bare wafer to a main etching chamber; and
turning on a plasma power source to perform the etching process on the photoresist.

9. The method of claim 1, wherein before the step of transporting the wafer to the main etch chamber further includes:
transporting the bare wafer from a port to a vacuum chamber; and
transporting the bare wafer from the vacuum chamber to a pre-alignment chamber.
